(19) United States
(12) Patent Application Publication
(10) Pub. No.: US 2012/0238037 A1
(43) Pub. Date: Sep. 20, 2012

(54) IMMUNOLOGICAL ASSAY AND IMMUNOLOGICAL ASSAY KIT

(75) Inventor: Masaaki Kobayashi, Kawasaki-shi (JP)

(73) Assignee: CANON KABUSHIKI KAISHA, Tokyo (JP)

(21) Appl. No.: 13/497,795

(22) PCT Filed: Oct. 15, 2010
(86) PCT No.: PCT/JP2010/068642

§ 371 Date: Mar. 22, 2012

(30) Foreign Application Priority Data
Oct. 27, 2009 (JP) 2009-247127

(51) Int. Cl. G01N 33/566; G01N 21/64; G01N 33/577

(52) U.S. Cl. 436/501

(57) ABSTRACT

An antibody enabling a rapid measurement of HMGB1, for example a combination of antibodies to HMGB1, is elucidated and an immunological assay and an immunological assay kit are provided. It is intended to provide an immunological assay kit for determining the presence or absence, or the concentration, or both of HMGB1, including a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B).

### TECHNICAL FIELD

The present invention relates to a diagnostic method for monitoring the severity of sepsis and sepsis-related symptoms. The diagnostic method includes an immunological detection method and an immunological assay kit therefor to rapidly assay HMGB1, which is a candidate marker for diseases such as sepsis.

### BACKGROUND ART

High Mobility Group Box 1 (hereinafter, may be abbreviated as “HMGB1”) is a major component of a non-histone nuclear protein. HMGB1 is a protein of approximately 30 kDa, which is known as a transcription regulatory factor. In 1999, Wang, et al. showed that HMGB1 was a candidate marker for sepsis.

PTL 1 proposes a method of measuring a serum concentration of HMGB1 as a diagnostic method for monitoring the severity of sepsis or septic shock, or a possible lethality rate.

Further, PTL 2 proposes an antibody specifically binding to human HMGB1 and an immunological assay and an immunological assay kit for human HMGB1 using the antibody. Furthermore, HMGB1 ELISA Kit II (trade name, Shino-Test Corporation) and HMGB1 (human) Detection Set (trade name, Apotech Corporation), which are research reagents for measuring a concentration of HMGB1 in human serum and plasma, are commercially available.

PTL 1 proposes diagnostic and prognostic methods in second claim 7 (PTL1 has two claim 7 by mistake). The methods are for monitoring the severity of sepsis and sepsis-related symptoms in patients exhibiting shock-like symptoms associated with symptoms characterized by activation of the inflammatory cascade or in patients at risk of exhibiting such symptoms, and predicting possible clinical courses.

PTL 2 discloses an invention relating to an antibody prepared using a peptide having an amino acid sequence represented by GKGDPKKPRGK (SEQ ID NO: 1) as an immunogen. The above patent application also discloses an invention relating to an antibody prepared using a peptide having an amino acid sequence obtained by subjecting the amino acid sequence represented by GKGDPKKPRGK (SEQ ID NO: 1) to deletion, substitution, insertion, or addition, or modification of one to several amino acid residues as an immunogen. This patent document further discloses an invention relating to an immunological assay and an immunological assay kit for human HMGB1 using these antibodies.

Meanwhile, HMGB1 was suggested to be associated with the lethality rate in the event of sepsis as it exhibits cytotoxicity when released from nuclei in the event of sepsis and appears later than TNF-α and IL-1β, which are the conventional inflammatory cytokines.

PTL 1 (Table 2) shows a correlation between a serum concentration of HMGB1 and cases of death, reporting that fatal cases were observed in patients reaching the maximum serum level of HMGB1.

Further, PTL 1 (paragraph [0012]) reports that the serum level of HMGB1 is elevated approximately 16 hours later than the commonly known initiation mediators of sepsis (for example, TNF and IL-1), and then reaches the plateau level.

That is, it can be understood that HMGB1 is not a simple marker but HMGB1 per se is a crucial mediator of organ damage in the event of septic shock. Thus, it is important to measure HMGB1 in a shorter time rather than measuring it as a marker.

No specific description pertaining to the assay time is found in the diagnostic assay described in PTL 1. Meanwhile, the antibody described in PTL 2 is an antibody prepared for the purpose of providing an antibody specifically binding to human HMGB1, not an antibody prepared for the purpose of rapidly measuring human HMGB1. Accordingly, there has been difficulty in utilizing the antibodies described in PTL 1 and PTL 2 for the purpose of rapidly measuring HMGB1.

Further, there has also been difficulty in utilizing commercially available research reagents, HMGB1 ELISA Kit II and HMGB1 (human) Detection Set, for the same purpose because it takes more than one night to obtain the results of an assay with these reagents.

Summing up the above, while it is a publicly known fact that a rapid measurement of HMGB1 is important for promptly monitoring the severity of sepsis and sepsis-related symptoms, neither immunological assay nor immunological assay kit enabling the above has been reported.

### CITATION LIST

### Patent Literature

PTL 1: Japanese Patent Application Laid-Open No. 2003-520763

PTL 2: Japanese Patent Application Laid-Open No. 2003-096099

### SUMMARY OF INVENTION

The present invention aims to elucidate an antibody enabling a rapid measurement of HMGB1, for example a combination of antibodies to HMGB1, and provide an immunological assay and an immunological assay kit.

The present inventors carried out an intensive research. As a result, they have found an immunological assay and an immunological assay kit therefor for determining the presence or absence, or the concentration, or both of HMGB1 using a combination of a first antibody to be immobilized on a carrier and a second antibody to be modified with a labeling substance, which are selected from particular antibodies.

An immunological assay for determining the presence or absence, or the concentration, or both of HMGB1 present in a sample uses a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B).

Antibody (A):

Antibody (A) is a polyclonal antibody specific for human HMGB1 wherein antibody (A) is produced by and obtained from a rabbit immunized with a protein, as an immunogen, obtained by modifying a peptide represented by

(SEQ ID NO: 2)
GKGDPKKPRGKMSSYA

with KLH (Keyhole Limpet Hemocyanin).

Antibody (B):

Antibody (B) is a polyclonal antibody specific for human HMGB1 wherein antibody (B) is produced by and obtained from a rabbit immunized with a protein, as an immunogen, represented by

(SEQ ID NO: 3)
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEDEEDEEDEEEE
EDEEDEDEEEDDDDE.

Antibody (C):

Antibody (C) is a monoclonal antibody specific for human HMGB1 wherein antibody (C) is produced by and obtained from a mouse immunized with a protein, as an immunogen, obtained by modifying a polypeptide represented by

(SEQ ID NO: 4)
MGKGDPKKPRGKMSSYAFFVQTCREEHKKKHPDASVNFSEFSKKCSERWK
TMSAKEKGKFEDMAKADKARYEREMKTYIPPKGETKKKFK

with a GST (glutathione-S-transferase) tag.

Antibody (D):

Antibody (D) is a monoclonal antibody specific for human HMGB1 wherein antibody (D) is produced by and obtained from a mouse immunized with a protein, as an immunogen, obtained by modifying a protein represented by

(SEQ ID NO: 5)
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEDEEDEEDEEEE
EDEEDEDEEEDDDDE

with a GST (glutathione-S-transferase) tag.

Further, an immunological assay kit for determining the presence or absence, or the concentration, or both of HMGB1 present in a sample includes a first antibody and a second antibody, wherein the first antibody is immobilized on a carrier and the second antibody is modified with a labeling substance, and the first antibody and the second antibody are any of the antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B).

The present immunological assay and the present immunological assay kit use, from among commercially available antibodies to HMGB1, such a combination of the first antibody and the second antibody that enables a rapid measurement of HMGB1. This enables shortening of the measurement time of HMGB1 to several tens of minutes, while the measurement of HMGB1 has conventionally required nearly one day.

### BRIEF DESCRIPTION OF DRAWINGS

FIG. 1 is a diagram illustrating rapid measurements of HMGB1 using the immunological assay and the immunological assay kit according to a first Example of the present invention.

FIG. 2 is a diagram comparing rapid measurements of HMGB1 using the immunological assay and the immunological assay kit according to a first Example of the present invention.

### DESCRIPTION OF EMBODIMENTS

Hereinafter, the embodiments of the present invention will be described in detail. It is to be noted that the embodiments individually disclosed below are examples of the immunological assay and the immunological assay kit of the present invention. The present invention is not limited to these examples.

### First Embodiment

The first embodiment of the present invention is an immunological assay for determining the presence or absence, or the concentration, or both of HMGB1 using a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B).

Antibody (A):

Antibody (A) is a polyclonal antibody specific for human HMGB1 wherein antibody (A) is produced by and obtained from a rabbit immunized with a protein, as an immunogen, obtained by modifying a peptide represented by

(SEQ ID NO: 2)
GKGDPKKPRGKMSSYA

with KLH (Keyhole Limpet Hemocyanin).

Antibody (B):

Antibody (B) is a polyclonal antibody specific for human HMGB1 wherein antibody (B) is produced by and obtained from a rabbit immunized with a protein, as an immunogen, represented by

(SEQ ID NO: 3)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE.

Antibody (C):

Antibody (C) is a monoclonal antibody specific for human HMGB1 wherein antibody (C) is produced by and obtained from a mouse immunized with a protein, as an immunogen, obtained by modifying a polypeptide represented by

(SEQ ID NO: 4)
MGKGDPKKPRGKMSSYAFFVQTCREEHKKKHPDASVNFSEFSKKCSERW
KTMSAKEKGKFEDMAKADKARYEREMKTYIPPKGETKKKFK

with a GST (glutathione-S-transferase) tag.

Antibody (D):

Antibody (D) is a monoclonal antibody specific for human HMGB1 wherein antibody (D) is produced by and obtained from a mouse immunized with a protein, as an immunogen, obtained by modifying a protein represented by

(SEQ ID NO: 5)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE

with a GST (glutathione-S-transferase) tag.

### Second Embodiment

The second embodiment of the present invention is an immunological assay kit for determining the presence or absence, or the concentration, or both of HMGB1, including a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B).

Hereinbelow, the first and the second embodiments of the present invention will be described in detail.

(1) Immunological Assay

The immunological assay of the present invention is an immunological assay for measuring HMGB1 contained in a sample utilizing an antigen-antibody reaction, wherein the aforementioned combination of the first antibody and the second antibody is such a combination that enables a rapid measurement of HMGB1. Specifically, the combination is any one of the aforementioned six combinations of antibodies (A) and (B), antibodies (A) and (C), antibodies (B) and (A), antibodies (B) and (C), antibodies (B) and (D), and antibodies (C) and (B).

That is, the immunological assay of the present invention is an immunological assay for measuring HMGB1, using any one of the aforementioned six combinations as a combination of the first antibody binding to human HMGB1, which is a substance to be measured, and the second antibody. According to the above, the immunological assay of the present invention enables a rapid measurement of HMGB1 contained in a sample.

Examples of the immunological assay include an Enzyme-Linked Immunosorbent Assay (ELISA, EIA), a fluorescent immunoassay (FIA), a radioimmunoassay (RIA), a luminescence immunoassay (LIA), an enzyme antibody technique, a fluorescence antibody assay, an immunochromatography, an immunonephelometry, a latex turbidimetry, and a latex agglutination assay.

Further, the measurement in the immunological assay of the present invention can be performed manually or using an apparatus such as an analytical apparatus.

Further, the immunological assay of the present invention can be operated according to a publicly known method. For example, the first antibody immobilized on a carrier, a sample, and the second antibody modified with a labeling substance are reacted simultaneously or in sequence. A complex of “the first antibody immobilized on a carrier-HMGB1-the second antibody modified with a labeling substance” is formed by the above reaction, and the amount (concentration) of HMGB1 contained in the sample can be measured based on the amount of the second antibody modified with a labeling substance contained in the complex.

For example, an enzyme-linked immunosorbent assay may be carried out using a microplate on which the first antibody is immobilized, a diluted solution of specimen, the second antibody modified with an enzyme such as HRP, a wash buffer, and a substrate solution. Further, the measurement may be performed by allowing the enzyme modifying the second antibody to react with its substrate under the optimum conditions for the enzyme and measuring the amount of the product of the enzyme reaction by an optical method and the like.

Also, a fluorescent immunoassay may be performed using an optical waveguide on which the first antibody is immobilized, a diluted solution of specimen, the second antibody modified with a fluorescent substance, and a wash buffer. Also, the measurement may be performed by irradiating the fluorescent substance modifying the second antibody with the excitation light, and measuring the intensity of the fluorescence emitted by the fluorescent substance.

Further, when a radioimmunoassay is carried out, the amount of radiation emitted by a radioactive substance is measured. Also, when a luminescence immunoassay is carried out, the amount of light emitted from a luminescent reaction system is measured.

Further, when an immunonephelometry, a latex turbidimetry, and a latex agglutination assay, and the like are carried out, the transmitted light and the scattering light are measured by an endpoint method or a rate method. Also, when an immunochromatography and the like are visually carried out, the color of a labeling substance appearing on the test line is visually measured. It is to be noted that an instrument such as an analytical apparatus can be used instead of the visual measurement.

(2) Sample to be Measured

The sample to be used in the immunological assay of the present invention includes all of the biological samples possibly containing HMGB1 such as a body fluid including blood, serum, plasma, urine, semen, spinal fluid, saliva, sweat, tear, ascites or amniotic fluid of a human.

(3) Antibody

Antibody (A) in the immunological assay and the immunological assay kit of the present invention may be a polyclonal antibody specific for human HMGB1 wherein antibody (A) is produced by and obtained from a rabbit immunized with a protein, as an immunogen, obtained by modifying a peptide represented by

GKGDPKKPRGKMSSYA (formula I)

with KLH (Keyhole Limpet Hemocyanin). Rabbit polyclonal to HMGB1-Aminoterminal end (trade name, Code ab67281) sold by Abcam plc. can be used. It is to be noted that an antibody specific for KLH has been desirably removed.

Further, antibody (A) also includes a polyclonal antibody specific for human HMGB1 wherein antibody (A) is produced by and obtained from a rabbit immunized with a protein, as an immunogen, obtained by modifying a peptide having an amino acid sequence obtained by subjecting the amino acid sequence expressed by formula (I) to deletion, substitution, insertion, or addition, or modification of one to several amino acid residues with KLH (Keyhole Limpet Hemocyanin).

Further, antibody (B) in the immunological assay and the immunological assay kit of the present invention may be a polyclonal antibody specific for human HMGB1 wherein antibody (B) is produced by and obtained from a rabbit immunized with a protein, as an immunogen, represented by MGKGDPKKPRGKMSSYAFFVQTCREEHKKKHPDASVNFSEFSKKCSERWKTMSAK EKGKFEDMAKADKARYEREMKTYIPPKGETKKKFKDPNAPKRPPSAFFLFCSEYR PKIKGEHPGLSIGDVAKKLGEMWNNTAADDKQPYEKKAAKLKEKYEKDIAAYRAK GKPDAAKKGVVKAEKSKKKKEEEEDEEDEEDEEEEEDEEDEDEEEDDDDE (SEQ ID NO:3). Antibody to HMGB1 (trade name, Cat. No. 10829-1-AP) sold by Proteintech Group, Inc. can be used.

Antibody (B) also includes a polyclonal antibody specific for human HMGB1 wherein antibody (B) is produced by and obtained from a rabbit immunized with a protein, as an immunogen, having an amino acid sequence obtained by subjecting the amino acid sequence expressed by formula (II) to deletion, substitution, insertion, or addition, or modification of one to several amino acid residues.

Further, antibody (C) in the immunological assay and the immunological assay kit of the present invention may be a monoclonal antibody specific for human HMGB1 wherein antibody (C) is produced by and obtained from a mouse immunized with a protein, as an immunogen, obtained by modifying a polypeptide represented by

(SEQ ID NO: 4)
MGKGDPKKPRGKMSSYAFFVQTCREEHKKKHPDASVNFSEFSKKCSERW
KTMSAKEKGKFEDMAKADKARYEREMKTYIPPKGETKKKFK

with a GST (glutathione-S-transferase) tag. HMGB1 monoclonal antibody (M08), Clone 2F6 (trade name, Cat. No. H003146-M08) sold by Abnova Corporation can be used. It is to be noted that an antibody specific for GST has been desirably removed.

Further, antibody (C) also includes a monoclonal antibody specific for human HMGB1 wherein antibody (C) is produced by and obtained from a mouse immunized with a protein, as an immunogen, obtained by modifying a peptide having an amino acid sequence obtained by subjecting the amino acid sequence expressed by formula (III) to deletion, substitution, insertion, or addition, or modification of one to several amino acid residues with a GST (glutathione-S-transferase) tag.

Further, antibody (D) in the immunological assay and the immunological assay kit of the present invention may be a monoclonal antibody specific for human HMGB1 wherein antibody (D) is produced by and obtained from a mouse immunized with a protein, as an immunogen, obtained by modifying a protein represented by

(SEQ ID NO: 5)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE

with a GST (glutathione-S-transferase) tag. HMGB1 monoclonal antibody (MO2), Clone 1D5 (trade name, Cat. No. H003146-MO2) sold by Abnova Corporation can be used.

Further, antibody (D) also includes a monoclonal antibody specific for human HMGB1 wherein antibody (D) is produced by and obtained from a mouse immunized with a protein, as an immunogen, obtained by modifying a peptide having an amino acid sequence obtained by subjecting the amino acid sequence expressed by formula (IV) to deletion, substitution, insertion, or addition, or modification of one to several amino acid residues with a GST (glutathione-S-transferase) tag.

(4) First Antibody

The first antibody in the immunological assay and the immunological assay kit of the present invention is immobilized on a carrier. That is, the first antibody is prepared by allowing one of the antibodies (A), (B), and (C) to adsorb or bind to a carrier through physisorption, chemical binding, or a method such as a combination of these.

The antibody immobilized by physisorption can be prepared according to a publicly known method. Examples of such a method include a method in which the antibody and a carrier are mixed and contacted with each other in a solution such as a buffer and a method in which the antibody dissolved in a buffer and the like is allowed to contact a carrier.

Further, the antibody immobilized by chemical binding can also be prepared according to a publicly known method. Examples of such a method include a method in which the antibody and a carrier are mixed with a divalent cross-linking reagent such as glutaraldehyde, carbodiimide, imide ester, or maleimide and contacted with each other so that amino groups, carboxyl groups, thiol groups, aldehyde groups, hydroxyl groups, or the like of both of the antibody and the carrier react.

Further, a treatment for inhibiting a non-specific reaction, spontaneous agglomeration of the carrier on which the antibody is immobilized, and the like can be performed according to a publicly known method, if needed. Examples of such a method include a method in which the surface or the inner surface of the carrier on which the antibody is immobilized is contacted with a protein such as bovine serum albumin (BSA), casein, gelatin, egg albumin, or a salt thereof, a surfactant, powdered skim milk, or the like so that the surface or the inner surface of the carrier is covered with these substances.

(5) Second Antibody

The second antibody in the immunological assay and the immunological assay kit of the present invention is modified with a labeling substance. The second antibody is prepared by allowing one of the antibodies (A), (B), (C) and (D) to adsorb or bind to a labeling substance through physisorption, chemical binding, or a method such as a combination of these.

The antibody having a labeling substance bound thereto by physisorption can be prepared according to a publicly known method. Examples of such a method include a method in which the antibody and a labeling substance are mixed and contacted with each other in a solution such as a buffer and a method in which the antibody dissolved in a buffer and the like is allowed to contact a labeling substance.

For example, when the labeling substance is gold colloid or latex, physisorption is effective. An antibody labeled with gold colloid is obtainable by mixing the antibody and gold colloid in a buffer and allowing them to contact each other.

Further, the antibody modified with a labeling substance by chemical binding can also be prepared according to a publicly known method. Examples of such a method include a method in which the antibody and a labeling substance are mixed with a divalent cross-linking reagent such as glutaraldehyde, carbodiimide, imide ester, or maleimide and contacted with each other so that amino groups, carboxyl groups, thiol groups, aldehyde groups, hydroxyl groups, or the like of both of the antibody and the labeling substance react. For example, when the labeling substance is a fluorescent substance, an enzyme, or a chemiluminescent substance, chemical binding is effective.

Further, a treatment for inhibiting a non-specific reaction, spontaneous agglomeration of the antibody modified with labeling substances, and the like can be performed according to a publicly known method, if needed. Examples of such a method include a method in which the antibody having a labeling substance bound thereto is contacted with a protein such as bovine serum albumin (BSA), casein, gelatin, egg albumin, or a salt thereof, a surfactant, powdered skim milk, or the like so that the antibody is covered with these substances.

Also, when an enzyme-linked immunosorbent assay is carried out, peroxidase (POD), alkaline phosphatase (ALP), β-galactosidase, urease, catalase, glucose oxidase, lactate dehydrogenase, amylase, or the like can be used as a labeling substance.

Also, when a fluorescent immunoassay is carried out, fluorescein isothiocyanate, tetramethylrhodamine isothiocyanate, substituted rhodamine isothiocyanate, dichlorotriazine isothiocyanate, cyanine, merocyanine, or the like can be used.

Also, when a radioimmunoassay is carried out, tritium, iodine-125, iodine-131, or the like can be used.

Also, when a luminescence immunoassay is carried out, a luminol compound, a luciferase compound, an acridinium ester, a dioxetane compound, or the like can be used.

Also, when an immunochromatography, an immunonephelometry, a latex turbidimetry, and a latex agglutination assay are carried out, particles made of a material such as polystyrene, a styrene-styrene sulfonate copolymer, an acrylonitrile-butadiene-styrene copolymer, a vinyl chloride-acrylic acid ester copolymer, a vinyl acetate-acrylic acid copolymer, polyacrolein, a styrene-methacrylic acid copolymer, a styrene-glycidyl (meth)acrylic acid copolymer, a styrene-butadiene copolymer, a (meth)acrylic acid polymer, an acrylic acid polymer, latex, gelatin, a liposome, a microcapsule, silica, alumina, carbon black, a metal compound, metal, metal colloid, a ceramic, or a magnetic material can be used.

(6) Carrier

As the carrier in the immunological assay of the present invention, a solid carrier in the form of a bead made of a material such as polystyrene, polycarbonate, polyvinyl toluene, polypropylene, polyethylene, polyvinyl chloride, nylon, polymethacrylate, polyacrylamide, latex, a liposome, gelatin, agarose, cellulose, sepharose, glass, metal, a ceramic, or a magnetic material, a microplate, a test tube, a stick, a membrane, a specimen piece, or the like can be used. Specifically, as the carrier of the present invention, a waveguide configured as an optical waveguide can be used.

(7) Immunological Assay Kit

The immunological assay kit of the present invention is an immunological assay kit for measuring HMGB1 contained in a sample utilizing an antigen-antibody reaction, wherein the aforementioned first and second antibodies are at least one of the permutation combinations of antibody (A) and antibody (B), antibody (A) and antibody (C), antibody (B) and antibody (A), antibody (B) and antibody (C), antibody (B) and antibody (D), and antibody (C) and antibody (B). The immunological assay kit can be used for the aforementioned immunological assay of the present invention. Accordingly, a similar measurement principle and the like to the aforementioned immunological assay apply to the immunological assay kit of the present invention.

(8) Other Reagent Components in the Immunological Assay Kit

In the immunological assay kit of the present invention, various aqueous solvents can be used as a solvent. Examples of the aqueous solvent can include purified water, physiological saline, or various buffers such as a tris buffer, a phosphate buffer, or a phosphate-buffered physiological saline. No particular limitation is imposed on a pH of these buffers, and a suitable pH may be appropriately selected; however, the pH is generally selected within a range of pH 3 to 12.

Further, the immunological assay kit of the present invention may appropriately include, in addition to the aforementioned first antibody immobilized on a carrier and the second antibody modified with a labeling substance, one or more kinds of a protein such as bovine serum albumin (BSA), human serum albumin (HSA), casein, or a salt thereof, various salts, various sugars, powdered skim milk, sera of various animals such as normal rabbit serum, various preservatives such as sodium azide and an antibiotic, an activator, a reaction promoter, a sensitivity enhancer such as polyethylene glycol, a non-specific reaction inhibitor, various surfactants such as a non-ionic surfactant, an ampholytic surfactant, or an anionic surfactant, and the like. Although no particular limitation is imposed on the concentrations of these substances in an assay reagent, the concentrations can be 0.001 to 10% (W/V), can particularly be 0.01 to 5% (W/V).

(9) Composition of the Immunological Assay Kit

No particular limitation is imposed on the immunological assay kit of the present invention as long as the aforementioned first and second antibodies include at least one of antibody (A) and antibody (B), antibody (A) and antibody (C), antibody (B) and antibody (A), antibody (B) and antibody (C), and antibody (C) and antibody (B).

Further, the immunological assay kit of the present invention for sale may include, in addition to a reagent containing the aforementioned two antibodies, other reagents in combination.

Examples of the aforementioned other reagents include a buffer, a diluted solution of sample, a diluted solution of reagent, a reagent containing a labeling substance, a reagent containing a substance generating a signal such as color development, a reagent containing a substance involved in generation of a signal such as color development, a reagent containing a substance for calibration, a reagent containing a substance used for accuracy control.

Then, the aforementioned other reagents and the immunological assay reagent of the present invention may be appropriately used and sold in various combinations, for example, the other reagents may be provided as a first reagent and the immunological assay reagent of the present invention may be provided as a second reagent, or the immunological assay reagent of the present invention may be provided as a first reagent and the aforementioned other reagents may be provided as a second reagent.

Also, while no particular limitation is imposed on the configuration of the immunological assay kit of the present invention, in order to perform a rapid measurement in a simple manner, the immunological assay kit of the present invention can be provided as an all-in-one diagnostic kit, in which the components of the immunological assay kit of the present invention are integrated. Although no particular limitation is imposed on the aforementioned all-in-one diagnostic kit, examples thereof include an ELISA kit, a fluorescent immunoassay kit, and an immunochromatography kit.

For example, a configuration of an ELISA kit includes a microplate on which the first antibody is immobilized, a diluted solution of specimen, the second antibody modified with an enzyme such as HRP, a wash buffer, a substrate solution, and the like.

Further, when a fluorescent immunoassay kit is provided, the kit includes an optical waveguide on which the first antibody is immobilized, a diluted solution of specimen, the second antibody modified with a fluorescent substance, a wash buffer, and the like.

Further, when an immunochromatography kit is provided, the following embodiment may be provided as an example. A membrane having the aforementioned first antibody immobilized on one end (downstream side) thereof is stored in a reaction cassette. Meanwhile, a developing solution is set on the other end (upstream side) of the membrane, and a pad having a substrate for the aforementioned labeling substance added thereto is arranged in the downstream side near where the developing solution is set, and a pad having the second antibody labeled with the aforementioned labeling substance is arranged in the intermediate part of the membrane.

### EXAMPLES

Hereinafter, the present invention will be further specifically described in detail with Examples; however, the present invention is not limited to these Examples.

### Comparative Example 1

Selection (1) of an Antibody to HMGB1 Enabling a Rapid Measurement of HMGB1

In Comparative Example 1, calibration ranges and detection limits of commercially available antibodies to HMGB1 were compared by a fluorescent immunoassay (sandwich method) in order to select a combination of antibodies enabling a rapid detection of HMGB1.

(1) Antibodies to HMGB1 Used in the Selection

A part of antibodies to HMGB1 used in the selection is listed in Table 1. Also, as the antibodies to HMGB1 listed in the Table, Mouse Anti-HMGB1, 2 Monoclonal antibody (trade name, Shino-test Corporation), HMGB1 monoclonal antibody (M08), Clone 2F6 (trade name, Abnova Corporation), HMGB1 monoclonal antibody (MO2), Clone 1D5 (trade name, Abnova Corporation), Rabbit polyclonal to HMGB1-Aminoterminal end (trade name, Abcam plc.), Rabbit polyclonal to HMGB1 (trade name, Abcam plc.), Mouse monoclonal [HAP46.5] to HMGB1 (trade name, Abcam plc.), Antibody to HMGB1 (Proteintech Group), and High mobility group protein 1 (human) Detection Set (Apotech Corporation) were used.

It is to be noted that the selection of a commercially available antibody was carried out focusing on an antibody produced by and obtained from various animals immunized with a peptide of N-terminal portion as an immunogen and an antibody produced by and obtained from various animals immunized with a full-length recombinant HMGB1 as an immunogen.

Also, as an HMGB1 antigen, HMGB1 supplied by ATGen Diagnostica, Biological Industries Ltd., Proteintech Group, and Shino-test Corporation was appropriately used

TABLE 1

| Denotation | Antibody used |
|---|---|
| (i) | Mouse Anti-HMGB1, 2 Monoclonal antibody |
| (ii) | HMGB1 monoclonal antibody (M08), clone 2F6 |
| (iii) | HMGB1 monoclonal antibody (M02), clone 1D5 |
| (iv) | Rabbit polyclonal to HMGB1 - Aminoterminal |
| | end |
| (v) | Rabbit polyclonal to HMGB1 |
| (vi) | Mouse monoclonal [HAP46.5] to HMGB1 |
| (vii) | Antibody to HMGB1 |
| (viii) | High mobility group protein 1 (human) |
| | Detection Set |

(2) Immobilization of the First Antibody to a Carrier

The following operation was carried out using the antibodies to HMGB1 (i) to (viii) listed in Table 1.

A waveguide (manufactured by Canon Chemicals Inc.), which was an optical waveguide to be used in a fluorescent immunoassay, was first prepared. Then, an antibody to HMGB1 was diluted in a phosphate-buffered physiological saline (pH 7.2) at 10 μg/mL, which was then dispensed into the special holders for the waveguide (manufactured by Canon Chemicals Inc.) at 100 μL each. The waveguide was then immersed in a solution contained in the holder and left to stand at 4° C. for one full day. Thereafter, the waveguide was removed and the surface thereof was washed with a phosphate-buffered physiological saline (pH 7.2). Subsequently, BSA diluted in a phosphate-buffered physiological saline (pH 7.2) at 5% was dispensed into the special holders for the waveguide at 100 μL each, where the waveguide was immersed and then left to stand at room temperature for two hours. Subsequently, the waveguide was removed and the surface thereof was washed with a phosphate-buffered physiological saline (pH 7.2). Lastly, the waveguide was dried and stored at 4° C.

(3) Labeling the Second Antibody with a Fluorescent Substance

Labeling of each of the antibodies to HMGB1 (the second antibody) listed in Table 1 with a fluorescent substance was performed using HiLyte Fluore (trademark) 647 Labeling Kit (trade name, manufactured by Dojindo Laboratories) according to the manufacturer's protocol.

Subsequently, the fluorescently-labeled second antibody thus obtained was prepared at 5 μg/mL with a 1% BSA-containing phosphate-buffered physiological saline (pH 7.2) and stored at 4° C.

(4) Selection of a Combination of the Antibodies to HMGB1 Enabling a Rapid Measurement of HMGB1

For selecting a combination of the antibodies to HMGB1 enabling a rapid measurement of HMGB1, a fluorescent immunoassay apparatus PR610-2 (trade name, manufactured by Research International, Inc.) was used.

Specifically, a waveguide on which the first antibody was immobilized was inserted in the special assay holders for PR610-2. Also, 100 μL of 5 μg/mL of the fluorescently-labeled second antibody was dispensed into the assay holders. Further, using a 5% BSA-containing phosphate-buffered physiological saline (pH 7.2), solutions of HMGB1 antigen having eight different concentrations (10000, 1000, 100, 10, 1, 0.1, 0.01, and 0 ng/mL) or another eight different concentrations (1000, 250, 62.5, 15.6, 3.91, 0.98, 0.24, and 0 ng/mL) were prepared, and each of the antibody solutions was dispensed into the special holders at 100 μL each.

In the present Comparative Example, in order to select a combination of the antibodies enabling a rapid measurement of HMGB1, the reaction time of the waveguide on which the first antibody was immobilized and the HMGB1 antigen was set at 10 minutes and the reaction time of the waveguide on which the first antibody was immobilized and the fluorescently-labeled second antibody was set at five minutes.

Also, as the evaluation standard for selecting the antibody, a combination of antibodies whose calibration curve obtained by assaying at the aforementioned eight different concentrations of HMGB1 encompassed a calibration range of 1 to 100 ng/mL was determined to be acceptable.

The results of selection of a combination of antibodies to HMGB1 are shown in the column titled Evaluation 1 in Table 2. As a result, seven different combinations of the antibodies, namely Nos. 11, 12, 14, 16, 19, 22, and 43 passed the selection standard. Also, the above-described seven kinds of combinations of the antibodies were found to be combinations of two antibodies selected from four antibodies (denotations (ii), (iii), (iv), and (vii)).

TABLE 2

| | The first | The second | | |
|---|---|---|---|---|
| No. | antibody | antibody | Evaluation 1 | Evaluation 2 |
| 1 | (i) | (vi) | x | — |
| 2 | (iii) | (vi) | x | — |
| 3 | (viii) | (vi) | x | — |
| 4 | (iv) | (vi) | x | — |
| 5 | (v) | (vi) | x | — |
| 6 | (vii) | (vi) | x | — |
| 7 | (ii) | (vi) | x | — |
| 8 | (viii) | (iv) | x | — |
| 9 | (vi) | (iv) | x | — |
| 10 | (v) | (iv) | x | — |
| 11 | (vii) | (iv) | ∘ | ∘ |
| 12 | (ii) | (iv) | ∘ | x |
| 13 | (viii) | (ii) | x | — |
| 14 | (iv) | (ii) | ∘ | ∘ |
| 15 | (v) | (ii) | x | — |
| 16 | (vii) | (ii) | ∘ | ∘ |
| 17 | (vi) | (ii) | x | — |
| 18 | (viii) | (vii) | x | — |
| 19 | (iv) | (vii) | ∘ | ∘ |
| 20 | (v) | (vii) | x | — |
| 21 | (vi) | (vii) | x | — |
| 22 | (ii) | (vii) | ∘ | ∘ |
| 23 | (i) | (vi) | x | — |
| 24 | (i) | (iv) | x | — |
| 25 | (i) | (ii) | x | — |
| 26 | (i) | (vii) | x | — |
| 27 | (iii) | (vi) | x | — |
| 28 | (iii) | (iv) | x | — |
| 29 | (iii) | (ii) | x | — |
| 30 | (iii) | (vii) | x | — |
| 31 | (i) | (v) | x | — |
| 32 | (ii) | (v) | x | — |
| 33 | (iii) | (v) | x | — |
| 34 | (iv) | (v) | x | — |
| 35 | (vi) | (v) | x | — |
| 36 | (vii) | (v) | x | — |
| 37 | (viii) | (v) | x | — |
| 38 | (i) | (iii) | x | — |
| 39 | (ii) | (iii) | x | — |
| 40 | (iv) | (iii) | x | — |
| 41 | (v) | (iii) | x | — |
| 42 | (vi) | (iii) | x | — |
| 43 | (vii) | (iii) | ∘ | ∘ |
| 44 | (viii) | (iii) | x | — |

### Comparative Example 2

Selection (2) of an Antibody to HMGB1 Enabling a Rapid Measurement of HMGB1

In Comparative Example 2, the cross-reactivity between the seven different combinations of the antibodies selected in Comparative Example 1 (No. 11, 12, 14, 16, 19, 22, and 43) and HMGB2 was examined.

(1) Antibodies to HMGB1 Used in the Selection

For selection, each of the following three kinds of antibodies, namely HMGB1 monoclonal antibody (M08), Clone 2F6 (denotation (ii)), Rabbit polyclonal to HMGB1-Aminoterminal end (denotation (iv)), and Antibody to HMGB1 (denotation (vii)) was used as the first antibody. Also, each of the following four kinds of antibodies, namely HMGB1 monoclonal antibody (M08), Clone 2F6 (denotation (ii)), HMGB1 monoclonal antibody (M02), Clone 1D5 (denotation (iii)), Rabbit polyclonal to HMGB1-Aminoterminal end (denotation (iv)), and Antibody to HMGB1 (denotation (vii)) was used as the second antibody. Also, as an HMGB2 antigen, HMGB2 purified from HMG-1, -2 Mixture (trade name) supplied by Wako Pure Chemical Industries, Ltd. was used.

(2) Immobilization of the First Antibody to a Carrier

Three kinds of antibodies to HMGB1, namely those denoted by (ii), (iv), and (vii), were each diluted in a phosphate-buffered physiological saline (pH 7.2) at 10 μg/mL, which were then dispensed into the special holders for the waveguide at 100 μL each.

The waveguide was then immersed in each of the solutions of the antibodies to HMGB1 and left to stand at 4° C. for one full day. Thereafter, the waveguide was removed and the surface thereof was washed with a phosphate-buffered physiological saline (pH 7.2). Subsequently, BSA diluted in a phosphate-buffered physiological saline (pH 7.2) at 5% was dispensed into the special holders for the waveguide at 100 μL each, where the waveguide was immersed and then left to stand at room temperature for two hours. Subsequently, the waveguide was removed and the surface thereof was washed with a phosphate-buffered physiological saline (pH 7.2). Lastly, the waveguide was dried and stored at 4° C.

(3) Labeling the Second Antibody with a Fluorescent Substance

Labeling of the four kinds of antibodies to HMGB1, which are those denoted by (ii), (iii), (iv), and (vii), with a fluorescent substance was performed using HiLyte Fluore (trademark) 647 Labeling Kit (trade name, manufactured by Dojindo Laboratories) according to the manufacturer's protocol. Subsequently, the second antibody thus fluorescently-labeled was prepared at 5 μg/mL with a 1% BSA-containing phosphate-buffered physiological saline (pH 7.2) and then stored at 4° C.

(4) Confirmation of the Cross-Reactivity with HMGB2

For confirmation of the cross-reactivity with HMGB2, a fluorescent immunoassay apparatus PR610-2 was used.

Specifically, the waveguide on which the first antibody was immobilized was inserted in the special assay holders for PR610-2. Also, 100 μL of 5 μg/mL of the fluorescently-labeled second antibody was dispensed into the assay holders. Further, using a 5% BSA-containing phosphate-buffered physiological saline (pH 7.2), solutions of HMGB2 antigen having eight different concentrations (1000, 250, 62.5, 15.6, 3.91, 0.98, 0.24, and 0 ng/mL) were prepared, which were then dispensed into the special holders at 100 μL each.

In the present Comparative Example, the reaction time of the waveguide on which the first antibody was immobilized and the HMGB2 antigen was set at 10 minutes and the reaction time of the waveguide on which the first antibody was immobilized and the fluorescently-labeled second antibody was set at five minutes. Also, as the evaluation standard for selecting the antibody, such a combination of antibodies that emitted no signal in assays at any concentration of the aforementioned eight concentrations of HMGB2 was determined to be acceptable.

The results of examination of the cross-reactivity with HMGB2 were shown in the column titled Evaluation 2 in Table 2. As a result of examining seven different combinations of Nos. 11, 12, 14, 16, 19, 22, and 43, No. 12 exhibited a clear cross-reactivity with HMGB2.

In view of the foregoing, it was found that a combination of antibodies of No. 11, No. 14, No. 16, No. 19, No. 22, and No. 43 enabled a rapid measurement of HMGB1 without exhibiting a cross-reactivity with HMGB2.

### Example 1

Rapid measurement of HMGB1 by the Immunological Assay and the Immunological Assay Kit of the Present Invention

Using a combination of the antibodies (Nos. 11 and 19), which exhibited a favorable result among the permutation combinations of the antibodies to HMGB1 selected in Comparative Examples 1 and 2, a rapid measurement of HMGB1 was carried out.

(1) Immobilization of the First Antibody to a Carrier

The antibodies to HMGB1 denoted by (iv) and (vii) were each diluted in a phosphate-buffered physiological saline (pH 7.2) at 10 μg/mL, which were then dispensed into the special holders for the waveguide at 100 μl each. The waveguide was then immersed in each of the solutions of the antibodies to HMGB1 and left to stand at 4° C. for one full day. Thereafter, the waveguide was removed and the surface thereof was washed with a phosphate-buffered physiological saline (pH 7.2). Subsequently, BSA diluted in a phosphate-buffered physiological saline (pH 7.2) at 5% were dispensed into the special holders for the waveguide at 100 μL each, where the waveguide was immersed and left to stand at room temperature for two hours. Subsequently, the waveguide was removed and the surface thereof was washed with a phosphate-buffered physiological saline (pH 7.2). Lastly, the waveguide was dried and stored at 4° C.

(2) Labeling the Second Antibody with a Fluorescent Substance

Labeling of the antibodies to HMGB1 denoted by (iv) and (vii) with a fluorescent substance was performed using HiLyte Fluore (trademark) 647 Labeling Kit (trade name, manufactured by Dojindo Laboratories) according to the manufacturer's protocol.

Subsequently, the second antibody thus fluorescently-labeled was prepared at 5 μg/mL with a 1% BSA-containing phosphate-buffered physiological saline (pH 7.2) and then stored at 4° C.

(3) Rapid Measurement of HMGB1 by the Immunological Assay and the Immunological Assay Kit of the Present Invention

For a rapid measurement of HMGB1 by the immunological assay and the immunological assay kit of the present invention, a fluorescent immunoassay apparatus PR610-2 was used.

Specifically, the waveguide on which the antibody to HMGB1 denoted by one of (iv) and (vii) was immobilized was inserted into the special assay holders for PR610-2. Also, 100 μL of 5 μg/mL of the fluorescently-labeled antibody to HMGB1 denoted by one of (iv) and (vii) were dispensed into the assay holders. Further, using a 5% BSA-containing phosphate-buffered physiological saline (pH 7.2), solutions of HMGB1 antigen (Proteintech Group, Inc.) having 12 different concentrations (10 μg/mL to 1 μg/mL) were prepared and each of the antibody solutions was dispensed into the special holders at 100 μL each.

In the present Example, the reaction time of the waveguide on which the first antibody was immobilized and the HMGB1 antigen was set at 10 minutes and the reaction time of the waveguide on which the first antibody was immobilized and the fluorescently-labeled second antibody was set at five minutes. Further, the measurement was performed four times per HMGB1 antigen of each concentration.

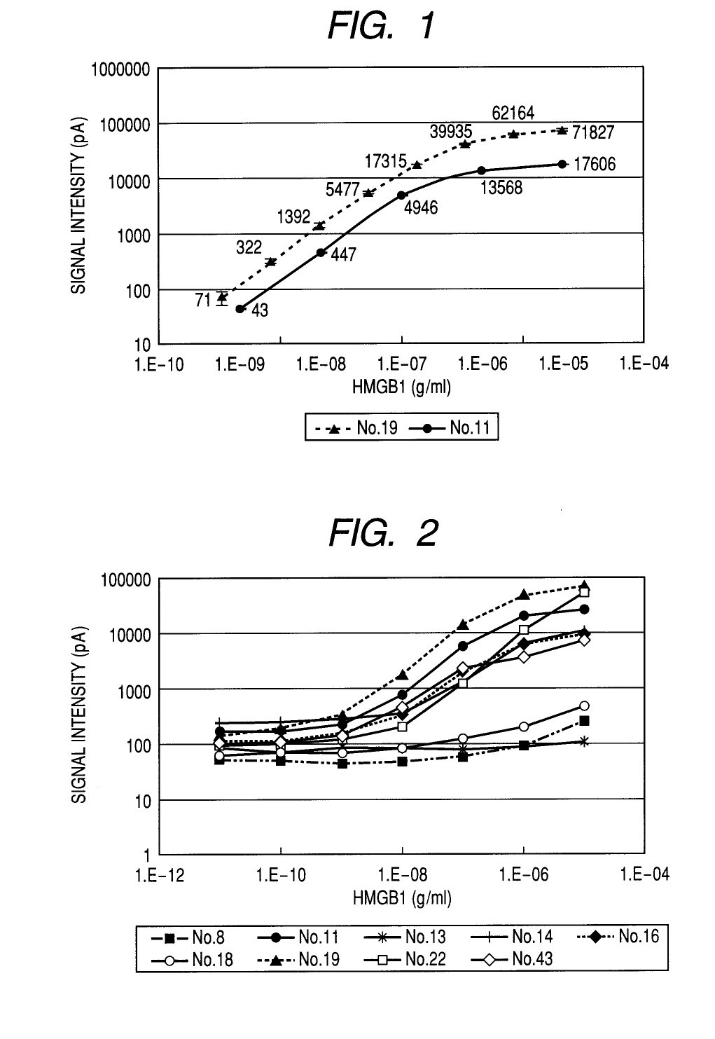

The calibration curves of a combination of the antibodies to HMGB1 (Nos. 11 and 19) are illustrated in FIG. 1. The calibration curves of No. 11 and No. 19 were found to have encompassed the calibration range of 1 to 100 ng/mL. Also, as to the assay time, the reaction time with the HMGB1 antigen was 10 minutes and the reaction time with the fluorescently-labeled second antibody was five minutes. Thus, even including the washing operation time consumed by the fluorescent immunoassay apparatus, the results of an assay of HMGB1 were obtained in approximately 20 minutes.

The above results were remarkably short compared to the results obtained with conventional assay kits.

(4) Evaluation of a Combination

Data comparing the combinations of the antibodies to HMGB1 of the present invention (No. 11, No. 12, No. 14, No. 16, No. 19, No. 22, and No. 43) and other combinations (No. 8, No. 13, and No. 18) is illustrated in FIG. 2.

It is understood that the combinations of the antibodies to HMGB1 of the present invention are remarkably sensitive to a change in concentration within a calibration range of 1 to 100 ng/mL in comparison to other combinations and that the combinations of the antibodies to HMGB1 of the present invention enable rapid and highly sensitive detection.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2009-247127, filed Oct. 27, 2009, which is hereby incorporated by reference herein in its entirety.

1. An immunological assay for determining the presence or absence, or the concentration, or both of HMGB1 using a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B):
Antibody (A) is a polyclonal antibody specific for human HMGB1 wherein antibody (A) is produced by and obtained from a rabbit immunized with a protein, as an immunogen, obtained by modifying a peptide represented by
(SEQ ID NO: 2)
GKGDPKKPRGKMSSYA
with KLH (Keyhole Limpet Hemocyanin);
Antibody (B) is a polyclonal antibody specific for human HMGB1 wherein antibody (B) is produced by and obtained from a rabbit immunized with a protein, as an immunogen, represented by
(SEQ ID NO: 3)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE;
Antibody (C) is a monoclonal antibody specific for human HMGB1 wherein antibody (C) is produced by and obtained from a mouse immunized with a protein, as an immunogen, obtained by modifying a polypeptide represented by
(SEQ ID NO: 4)
MGKGDPKKPRGKMSSYAFFVQTCREEHKKKHPDASVNFSEFSKKCSERW
KTMSAKEKGKFEDMAKADKARYEREMKTYIPPKGETKKKFK
with a GST (glutathione-S-transferase) tag; and
Antibody (D) is a monoclonal antibody specific for human HMGB1 wherein antibody (D) is produced by and obtained from a mouse immunized with a protein, as an immunogen, obtained by modifying a protein represented by
(SEQ ID NO: 5)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE
with a GST (glutathione-S-transferase) tag.

2. An immunological assay kit for determining the presence or absence, or the concentration, or both of HMGB1, comprising a first antibody and a second antibody, wherein the first antibody is immobilized on a carrier and the second antibody is modified with a labeling substance, and the first antibody and the second antibody are any of the following antibody (A), antibody (B), antibody (C), and antibody (D), and a permutation combination of the first antibody and the second antibody is any one of antibody (A) and antibody (B), antibody (A) and antibody (C), antibody (B) and antibody (A), antibody (B) and antibody (C), antibody (B) and antibody (D), and antibody (C) and antibody (B):
Antibody (A) is a polyclonal antibody specific for human HMGB1 wherein antibody (A) is produced by and obtained from a rabbit immunized with a protein, as an immunogen, obtained by modifying a peptide represented by
(SEQ ID NO: 2)
GKGDPKKPRGKMSSYA
with KLH (Keyhole Limpet Hemocyanin);
Antibody (B) is a polyclonal antibody specific for human HMGB1 wherein antibody (B) is produced by and obtained from a rabbit immunized with a protein, as an immunogen, represented by
(SEQ ID NO: 3)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE;
Antibody (C) is a monoclonal antibody specific for human HMGB1 wherein antibody (C) is produced by and obtained from a mouse immunized with a protein, as an immunogen, obtained by modifying a polypeptide represented by
(SEQ ID NO: 4)
MGKGDPKKPRGKMSSYAFFVQTCREEHKKKHPDASVNFSEFSKKCSERW
KTMSAKEKGKFEDMAKADKARYEREMKTYIPPKGETKKKFK
with a GST (glutathione-S-transferase) tag; and
Antibody (D) is a monoclonal antibody specific for human HMGB1 wherein antibody (D) is produced by and obtained from a mouse immunized with a protein, as an immunogen, obtained by modifying a protein represented by
(SEQ ID NO: 5)
MGKGDPKKPRGKMSSYAFFVQTCREEHKKKHPDASVNFSEFSKKCSERW
KTMSAKEKGKFEDMAKADKARYEREMKTYIPPKGETKKKFKDPNAPKRP
PSAFFLFCSEYRPKIKGEHPGLSIGDVAKKLGEMWNNTAADDKQPYEKK
AAKLKEKYEKDIAAYRAKGKPDAAKKGVVKAEKSKKKKEEEEDEEDEED
EEEEEDEEDEDEEEDDDDE
with a GST (glutathione-S-transferase) tag.

3. The immunological assay kit according to claim 2, wherein the carrier is a waveguide configured as an optical waveguide and the labeling substance is a fluorescent substance.


SEQUENCE LISTING (5 sequences)

SEQ ID NO: 1 - protein, 11 aa, Artificial
  Immunogen
GKGDPKKPRGK

SEQ ID NO: 2 - protein, 16 aa, Artificial
  Immunogen
GKGDPKKPRGKMSSYA

SEQ ID NO: 3 - protein, 215 aa, Artificial
  Immunogen
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 4 - protein, 90 aa, Artificial
  Immunogen
MGKGDPKKPRGKMSSYAFFVQTCREEHKKKHPDASVNFSEFSKKCSERWKTMSAKEKGKFEDMAKADKARYEREMKTYIPPKGETKKKFK

SEQ ID NO: 5 - protein, 215 aa, Artificial; identical to SEQ ID NO: 3
  Immunogen